(19) United States
(12) Patent Application Publication
(10) Pub. No.: US 2011/0207783 A1
(43) Pub. Date: Aug. 25, 2011

(54) RENIN INHIBITORS

(76) Inventors: Daniel Dube, Saint-Lazaire (CA); Austin Chen, Pierrefonds (CA); Daniel J. McKay, Chate Blondreau (CA)

(21) Appl. No.: 12/672,177

(22) PCT Filed: Aug. 4, 2008
(86) PCT No.: PCT/CA08/01429

§ 371 Date: Feb. 4, 2010

Related U.S. Application Data
(60) Provisional application No. 60/963,784, filed on Aug. 7, 2007.

(51) Int. Cl. A61K 31/44; C07C 233/60; A61K 31/165; C07D 209/26; A61K 31/4045; C07D 213/56; A61K 31/4406; C07D 213/73; A61P 9/00; A61P 13/12; A61P 11/00; A61P 25/28; A61P 25/22

(52) U.S. Cl. 514/352; 564/164; 514/620; 548/495; 514/419; 546/337; 514/357; 546/311

(57) ABSTRACT

The present invention relates to acyclic amino amide renin inhibitor compounds and their use in treating cardiovascular events and renal insufficiency.

### JOINT RESEARCH AGREEMENT

The claimed invention was made as a result of activities undertaken within the scope of a joint research agreement between Merck & Co., Inc. and Actelion Pharmaceuticals Ltd. The agreement was executed on Dec. 4, 2003. The field of the invention is described below.

### FIELD OF THE INVENTION

The invention relates to novel renin inhibitors of the general formula (I). The invention also concerns related aspects including processes for the preparation of the compounds, pharmaceutical compositions containing one or more compounds of formula (I) and especially their use as renin inhibitors in cardiovascular events and renal insufficiency.

### BACKGROUND OF THE INVENTION

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

The present invention relates to the identification of renin inhibitors of a non-peptidic nature and of low molecular weight. Described are orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis. So, the present invention describes these non-peptidic renin inhibitors.

The compounds described in this invention represent a novel structural class of renin inhibitors.

### SUMMARY OF THE INVENTION

The present invention is directed to certain compounds and their use in the inhibition of the renin enzyme, including treatment of conditions known to be associated with the renin system. The invention includes compounds of Formula I:

The present invention relates to compounds of the formula (I)

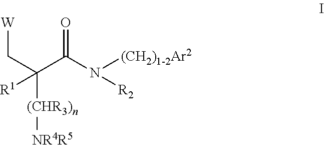

and pharmaceutically acceptable salts thereof, wherein
W is selected from the group consisting of
1) aryl, and
2) a heterocyclic ring system selected from the group consisting of:
a) a 5- or 6-membered saturated or unsaturated monocyclic ring with 1, 2, or 3 heteroatom ring atoms selected from the group consisting of N, O or S,
b) an 8-, 9- or 10-membered saturated or unsaturated bicyclic ring with 1, 2, or 3 heteroatom ring atoms selected from the group consisting of N, O or S, and
c) an 11- to 15-membered saturated or unsaturated triicyclic ring with 1, 2, 3, or 4 heteroatom ring atoms selected from the group consisting of N, O or S,
   wherein said aryl or heterocyclic ring is unsubstituted, mono-substituted with R^{w}, disubstituted with groups independently selected from R^{w}, trisubstituted with groups independently selected from R^{w}, or tetrasubstituted with groups independently selected from R^{w}, and wherein any stable S or N heterocyclic ring atom is unsubstituted or substituted with oxo, said hetercyclic ring R^{w} substitutions being on one or more heterocyclic ring carbon or nitrogen atoms;
R^{w} is selected from the group consisting of
   1) OH,
   2) NH₂,
   3) CN,
   4) OCF₂H,
   5) CF₃,
   6) C₁-C₃alkyl,
   7) C₁-C₃alkoxy,
   8) —SO₂C₁-C₃alkyl, and
   9) —SO₂H; and
n, in each instance in which it occurs, is independently 0, 1 or 2;
p, in each instance in which it occurs, is independently 0, 1 or 2;
R¹ and R³ are independently selected from the group consisting of H, C₁-C₆alkyl and
C₂-C₆alkenyl, wherein the alkyl and alkenyl group is unsubstituted or substituted with one, two, three or four substituents independently selected from:
   1) OH,
   2) CN,
   3) CF₃,
   4) COOH,
   5) C₁-C₆alkoxy,
   6) C(O)R^{b},
   7) C(O)N(R^{c})₂,
   8) S(O)ₚC₁-C₆alkyl,
   9) SO₂N(R^{c})₂,
   10) N(R^{c})₂,
   11) NHC(O)R^{b},
   12) NHC(O)NHR^{d},
   13) NHC(S)NHR^{d},
   14) NH(NR^{c})_{NHR}^{c},
   15) tetrazolyl, and
   16) —(CH₂)₁₋₂R^{e};
R⁴ is selected from the group consisting of H, C₁-C₆alkyl and C2-C6alkenyl, wherein the alkyl and alkenyl group is unsubstituted or substituted with one, two, three or four substituents independently selected from:
   1) OH,
   2) CN,
   3) CF₃,
   4) COOH,
   5) C₁-C₆alkoxy,
   6) C(O)R^{b},
   7) C(O)N(R^{c})₂,
   8) S(O)ₚC₁-C₆alkyl,
   9) SO₂N(R^{c})₂,
   10) N(R^{c})₂,
   11) NHC(O)R^{b},
   12) NHC(O)NHR^{d},
   13) NHC(S)NHR^{d},
   14) NH(NR^{c})NHR^{c}, and
   15) tetrazolyl,
      or R4, together with R5, forms a 5- or 6-membered heterocyclic ring which is unsubstituted or mono- or di-substituted with a substituent selected from the group consisting of ═O and C1-C6 alkyl;
R⁵ is selected from the group consisting of hydrogen and —C(NH(NH₂), or R⁵, together with R⁴, forms a 5- or 6-membered heterocyclic ring which is unsubstituted or mono- or di-substituted with a substituent selected from the group consisting of ═O and C1-C6 alkyl;
R² and R^{b} are independently selected from the group consisting of H, C₁-C₆alkyl, C₃-C₈cycloalkyl, C₂-C₆alkenyl, C₁-C₆alkoxy, CF₃ and CH₂CF₃;
R^{c} is selected from the group consisting of H, C₁-C₆alkyl and CH₂CF₃;
R^{d} is selected from the group consisting of H and C₁-C₆alkyl, wherein the alkyl group is unsubstituted or substituted with one, two, three or four substituents selected from the group consisting of:
   1) OH,
   2) CN,
   3) CF₃,
   4) COOH, and
   5) C(O)NHR^{c}, and
   6) tetrazolyl;
R^{e} is a 5- or 6-membered heteroaryl ring having 1 or 2 nitrogen atoms;
Ar² is independently selected from the group consisting of Ar¹ and a 9- or 10 membered fused bicyclic aryl or heteroaryl ring, wherein the fused bicyclic heteroaryl contains 1 to 4 heteroatoms selected from O, S and N, wherein the fused bicyclic aryl and heteroaryl are each unsubstituted or substituted with one, two, three or four substituents independently selected from the group consisting of:
   1) OH,
   2) CN,
   3) halogen,
   4) N₃,
   5) NO₂,
   6) COOH,
   7) OCF₂H,
   8) CF₃,
   9) C₁-C₆alkyl, unsubstituted or substituted with Ar³,
   10) C₁-C₆alkyl,
   11) C₂-C₆alkenyl,
   12) C₁-C₆alkoxy,
   13) C(O)C₁-C₆alkyl,
   14) S(O)ₚC₁-C₆alkyl,
   15) −O(CH₂)₁₋₂Ar³,
   16) —O(CH₂)₁₋₂D,
   17) —OC(O)D,
   18) —OC(O)NH(C₁-C₆alkylene)C(O)NH₂, and
   19) —OC(O)NH(C₁-C₆alkylene)(OH)R^{d};
      wherein substituents (10)-(14) are unsubstituted or substituted with one, two three or four substituents independently selected from the group consisting of:
   a) OH,
   b) COOR^{d},
   c) CN,
   d) CF₃,
   e) C₁-C₆alkoxy,
   f) S(O)ₚC₁-C₆alkyl,
   g) tetrazolyl
   h) —C(O)NH₂,
   i) —COON^{a},
   j) —NR^{d}R^{d}, and
   k) —NR^{d}C(O)R^{d};
      heteroaryl ring containing 1 to 3 heteroatoms selected from O, S and N, wherein the substituted aryl ring and substituted heteroaryl ring are substituted with one, two three or four substituents independently selected from the group consisting of:
   1) OH,
   2) CN,
   3) halogen,
   4) N₃,
   5) NO₂,
   6) COOH, 7) OCF₂H,
   8) CF₃,
   9) C₁-C₆alkyl,
   10) C₂-C₆alkenyl,
   11) C₁-C₆alkoxy,
   12) C(O)C₁-C₆alkyl, and
   13) S(O)ₚC₁-C₆alkyl,
      wherein substituents (9)-(13) are unsubstituted or substituted with one, two three or four substituents independently selected from the group consisting of:
   a) OH,
   b) COOH,
   c) CN,
   d) CF₃,
   e) C₁-C₆alkoxy,
   f) S(O)ₚC₁-C₆alkyl;
Ar¹ is an unsubstituted or substituted aryl ring or an unsubstituted or substituted 5- or 6-membered heteroaryl ring containing 1 to 3 heteroatoms selected from O, S and N, wherein the substituted aryl ring and substituted heteroaryl ring are substituted with one, two three or four substituents independently selected from the group consisting of:
   1) OH,
   2) CN,
   3) halogen,
   4) N₃,
   5) NO₂,
   6) COOH,
   7) OCF₂H,
   8) CF₃,
   9) C₁-C₆alkyl,
   10) C₂-C₆alkenyl,
   11) C₁ -C₆alkoxy,
   12) C(O)C₁-C₆alkyl, and
   13) S(O)ₚC₁-C₆alkyl,
      wherein substituents (9)-(13) are unsubstituted or substituted with one, two three or four substituents independently selected from the group consisting of:
   a) OH,
   b) COOH,
   c) CN,
   d) CF₃,
   e) C₁-C₆alkoxy,
   f) S(O)ₚC₁-C₆alkyl;
Ar³ is an unsubstituted or substituted aryl ring or an unsubstituted or substituted 5- or 6-membered heteroaryl ring containing 1 to 3 heteroatoms selected from O, S and N, wherein the substituted aryl ring and substituted heteroaryl ring are substituted with one, two three or four substituents independently selected from the group consisting of:
   1) OH,
   2) CN,
   3) OCF₂H,
   4) CF₃,
   5) C₁-C₃alkyl,
   6) C₁-C₃alkoxy, and
   7) —SO₂R^{d}; and
D is a 5- or 6-membered saturated heterocyclic ring having 1 or 2 nitrogen atoms and 0 or 1 oxygen atoms, wherein the ring may be unsubstituted or substituted with C₁-C₆alkyl,
   and optically pure enantiomers, mixtures of enantiomers such as racemates, diastereomers, mixtures of diastereomers, diastereomeric racemates, mixtures of diastereomeric racemates, meso-forms, tautomers, salts, solvates, and morphological forms thereof.

### DETAILED DESCRIPTION OF THE DISCLOSURE

In one embodiment of compounds of Formula I, W is phenyl, pyridyl, indolyl, or pyridyl substituted with NH₂, and all other variables are as previously defined.

In another embodiment of compounds of Formula I, R¹ is H, all other variables are as previously defined.

In another embodiment of compounds of Formula I, R³ is H, all other variables are as previously defined.

In another embodiment of compounds of Formula I, n is 1, R⁴ is H, and R⁵ is H, and all other variables are as previously defined

In another embodiment of compounds of Formula I, R² is cyclopropyl, and all other variables are as previously defined.

In another embodiment of compounds of Formula I, Ar² is phenyl which is disubstituted with a group independently selected from Cl, —CH₂CH₂OCH₃, —OCH₂CH₂OCH₃, and —CH₂CH₂CH₂OCH₃, and all other variables are as previously defined.

The compounds of Formula I above, and pharmaceutically acceptable salts thereof, are renin inhibitors. The compounds are useful for inhibiting renin and treating conditions such as hypertension.

Any reference to a compound of formula (I) is to be understood as referring also to optically pure enantiomers, mixtures of enantiomers such as racemates, diastereomers, mixtures of diastereomers, diastereomeric racemates, mixtures of diastereomeric racemates, meso-forms and tautomers, as well as salts (especially pharmaceutically acceptable salts) and solvates (including hydrates) of such compounds, and morphological forms, as appropriate and expedient. The present invention encompasses all these forms. Mixtures are separated in a manner known per se, e.g. by column chromatography, thin layer chromatography (TLC), high performance liquid chromatography (HPLC), or crystallization. The compounds of the present invention may have chiral centers, e.g. one chiral center (providing for two stereoisomers, (R) and (S)), or two chiral centers (providing for up to four stereoisomers, (R,R), (S,S), (R,S), and (S,R)). This invention includes all of these optical isomers and mixtures thereof. Unless specifically mentioned otherwise, reference to one isomer applies to any of the possible isomers. Whenever the isomeric composition is unspecified, all possible isomers are included.

Tautomers of compounds defined in Formula I are also included within the scope of the present invention. For example, compounds including carbonyl —CH₂C(O)— groups (keto forms) may undergo tautomerism to form hydroxyl —CH═C(OH)— groups (enol forms). Both keto and enol forms are included within the scope of the present invention.

In addition, compounds with carbon-carbon double bonds may occur in Z- and E-forms with all isomeric forms of the compounds being included in the present invention. Compounds of the invention also include nitrosated compounds of formula (I) that have been nitrosated through one or more sites such as oxygen (hydroxyl condensation), sulfur (sulfydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983). Salts are preferably the pharmaceutically acceptable salts of the compounds of formula (I). The expression “pharmaceutically acceptable salts” encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or, in case the compound of formula (I) is acidic in nature, with an inorganic base like an alkali or earth alkali base, e.g. sodium hydroxide, potassium hydroxide, calcium hydroxide and the like. For other examples of pharmaceutically acceptable salts, reference can be made notably to “Salt selection for basic drugs”, Int. J. Pharm. (1986), 33, 201-217.

The invention also includes derivatives of the compound of Formula I, acting as prodrugs. These prodrugs, following administration to the patient, are converted in the body by normal metabolic processes to the compound of Formula 1. Such prodrugs include those that demonstrate enhanced bioavailability (see Table 4 below), tissue specificity, and/or cellular delivery, to improve drug absorption of the compound of Formula I. The effect of such prodrugs may result from modification of physicochemical properties such as lipophilicity, molecular weight, charge, and other physicochemical properties that determine the permeation properties of the drug. The general terms used hereinbefore in formula I and hereinafter preferably have, within this disclosure, the following meanings, unless otherwise indicated. Where the plural form is used for compounds, salts, pharmaceutical compositions, diseases and the like, this is intended to mean also a single compound, salt, or the like.

The term “alkyl”, alone or in combination with other groups, means saturated, straight and branched chain groups with one to six carbon atoms, i.e., C₁₋₆ alkyl. Examples of alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred. Structural depictions of compounds may show a terminal methyl group as

“—CH₃”, “Me”, or

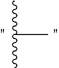

i.e., these have equivalent meanings.

The term “alkoxy”, alone or in combination with other groups, refers to an R—O— group, wherein R is an alkyl group. Examples of alkoxy groups are methoxy, ethoxy, propoxy, iso-propoxy, iso-butoxy, sec-butoxy and tert-butoxy.

The term “hydroxy-alkyl”, alone or in combination with other groups, refers to an HO—R— group, wherein R is an alkyl group. Examples of hydroxy-alkyl groups are HO—CH₂—, HO—CH₂CH₂—, HO—CH₂CH₂CH₂— and CH₃CH(OH)—.

The term “halogen” means fluorine, chlorine, bromine or iodine, preferably fluorine, chlorine or bromine, especially fluorine or chlorine.

The term “cycloalkyl”, alone or in combination, means a saturated cyclic hydrocarbon ring system with 3 to 8 carbon atoms, e.g. cyclopropyl, cyclobutyl, cyclopentyl, cyclohexyl, cycloheptyl and cyclooctyl.

The term “aryl” refers to aromatic mono- and poly-carbocyclic ring systems, also referred to as “arenes”, wherein the individual carbocyclic rings in the polyring systems are fused or attached to each other via a single bond. Suitable aryl groups include phenyl, naphthyl, indanyl and biphenylenyl. The abbreviation “Ph” represents phenyl.

Unless indicated otherwise, the term “heterocycle” (and variations thereof such as “heterocyclic” or “heterocyclyl”) broadly refers to (i) a stable 4- to 8-membered, saturated or unsaturated monocyclic ring, (ii) a stable 7- to 12-membered bicyclic ring system, or (iii) a stable 11- to 15-membered tricyclic ring system, wherein each ring in (ii) and (iii) is independent of, or fused to, the other ring or rings and each ring is saturated or unsaturated, and the monocyclic ring, bicyclic ring system or tricyclic ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the bicyclic and tricyclic ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

Saturated heterocyclics form a subset of the heterocycles. Unless expressly stated to the contrary, the term “saturated heterocyclic” generally refers to a heterocycle as defined above in which the entire ring system (whether mono- or poly-cyclic) is saturated. The term “saturated heterocyclic ring” refers to a 4- to 8-membered saturated monocyclic ring, a stable 7- to 12-membered bicyclic ring system, or a stable 11- to 15-membered tricyclic ring system, which consists of carbon atoms and one or more heteroatoms selected from N, O and S. Representative examples include piperidinyl, piperazinyl, azepanyl, pyrrolidinyl, pyrazolidinyl, imidazolidinyl, oxazolidinyl, isoxazolidinyl, morpholinyl, thiomorpholinyl, thiazolidinyl, isothiazolidinyl, and tetrahydrofuryl (or tetrahydrofuranyl).

Unsaturated heterocyclics form another subset of the heterocycles. Unless expressly stated to the contrary, the term “unsaturated heterocyclic” generally refers to a heterocycle as defined above in which the entire ring system (whether mono- or poly-cyclic) is not saturated, i.e., such rings are either unsaturated or partially unsaturated. Unless expressly stated to the contrary, the term “heteroaromatic ring” refers a 5- or 6-membered monocyclic aromatic ring, a 7- to 12-membered bicyclic ring system, or a 11- to 15-membered tricyclic ring system, which consists of carbon atoms and one or more heteroatoms selected from N, O and S. In the case of substituted heteraromatic rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

Representative examples of bicyclic heterocycles include benzotriazolyl, indolyl, isoindolyl, indazolyl, indolinyl, isoindolinyl, quinoxalinyl, quinazolinyl, cinnolinyl, chromanyl, isochromanyl, tetrahydroquinolinyl, quinolinyl, tetrahydroisoquinolinyl, isoquinolinyl, 2,3-dihydrobenzofuranyl, 2,3-dihydrobenzo-1,4-dioxinyl (i.e.,

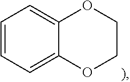

imidazo(2,1-b)(1,3)thiazole, (i.e.,

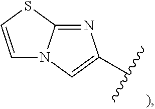

and benzo-1,3-dioxolyl (i.e.,

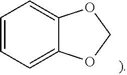

In certain contexts herein,

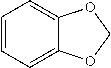

is alternatively referred to as phenyl having as a substituent methylenedioxy attached to two adjacent carbon atoms.

The term “heteroaryl”, alone or in combination, refers to certain heterocyclic rings which are six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing two heteroatoms independently selected from oxygen, nitrogen and sulfur and benzofused derivatives of such rings; five-membered aromatic rings containing three nitrogen atoms and benzofused derivatives thereof; a tetrazolyl ring; a thiazinyl ring; or coumarinyl. Examples of such ring systems are furanyl, thienyl, pyrrolyl, pyridinyl, pyrimidinyl, indolyl, quinolinyl, isoquinolinyl, imidazolyl, triazinyl, thiazolyl, isothiazolyl, pyridazinyl, pyrazolyl, oxazolyl, isoxazolyl, benzothienyl, quinazolinyl and quinoxalinyl.

Specific examples of compounds of formula I, and pharmaceutically acceptable salts thereof, include those listed below:

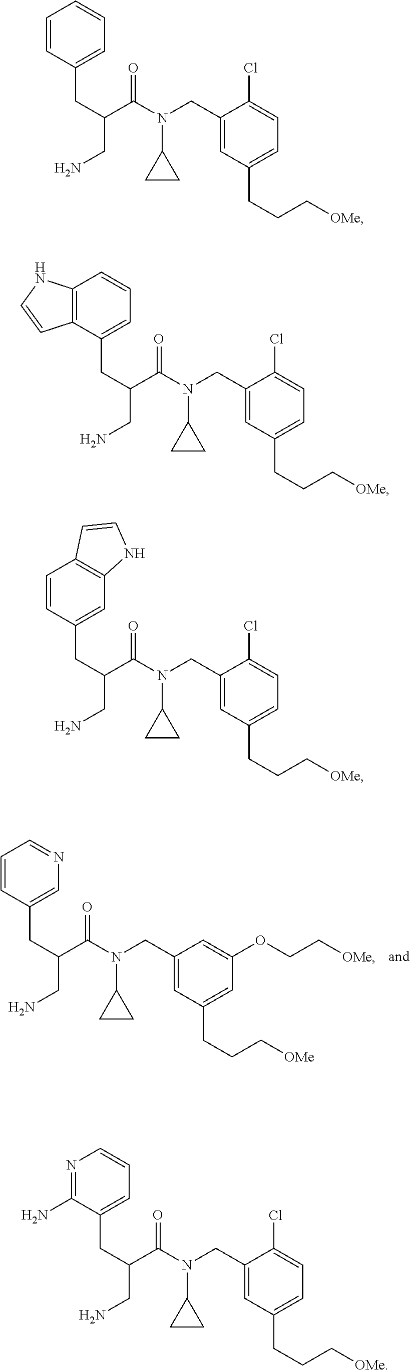

The present invention also encompasses a pharmaceutical formulation comprising a pharmaceutically acceptable carrier and the compound of Formula I or a pharmaceutically acceptable crystal form or hydrate thereof. A preferred embodiment is a pharmaceutical composition of the compound of Formula I, comprising, in addition, a second agent.

### List of Abbreviations:

ABTS 2,2′-Azino-bis(3-ethylbenzthiazoline-6-sulfonic Acid)⁻ 2NH₃
Boc t-butyloxycarbonyl
BSA bovine serum albumin
DME dimethoxyethane
DMF dimethylformamide
DMSO dimethylsulfoxide
EDTA ethylenediaminetetraacetic acid
EIA enzyme immunoassay
HATU O-(7-azabenzotriazol-1-yl)-N,N,N′,N′-tetramethyluronium hexafluorophosphate
Hex hexane
PBS phosphate-buffered saline
TFA trifluoroacetic acid
THF tetrahydrofuran

Unless expressly stated to the contrary, all ranges cited herein are inclusive. For example, an alkyl group described as C₁-C₆ alkyl means the alkyl group can contain 1, 2, 3, 4, 5 or 6 carbon atoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed.

In compounds of the invention having pyridyl N-oxide moieties, the pyridyl-N-oxide portion is structurally depicted using conventional representations such as which have equivalent meanings.

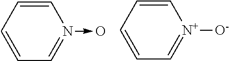

The invention relates to a method for the treatment and/or prophylaxis of diseases which are related to hypertension, congestive heart failure, pulmonary hypertension, systolic hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

Compounds of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

The term “administration” and variants thereof (e.g., “administering” a compound) in reference to a compound of Formula I mean providing the compound or a prodrug of the compound to the individual in need of treatment or prophylaxis. When a compound of the invention or a prodrug thereof is provided in combination with one or more other active agents (e.g., an agent such as anangiotensin II receptor antagonist, ACE inhibitor, or other active agent which is known to reduce blood pressure), “administration” and its variants are each understood to include provision of the compound or prodrug and other agents at the same time or at different times. When the agents of a combination are administered at the same time, they can be administered together in a single composition or they can be administered separately.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit renin and thereby elicit the response being sought (i.e., an “inhibition effective amount”). When the active compound (i.e., active ingredient) is administered as the salt, references to the amount of active ingredient are to the free form (i.e., the non-salt form) of the compound.

In a preferred embodiment, this amount is comprised between 1 mg and 1000 mg per day. In a particularly preferred embodiment, this amount is comprised between 1 mg and 500 mg per day. In a more particularly preferred embodiment, this amount is comprised between 1 mg and 200 mg per day.

In the method of the present invention (i.e., inhibiting renin), the compounds of Formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in reference such as Fieser and Fieser's Reagents for Organic Synthesis; Wiley & Sons: New York, Volume 1-21; R. C. Larock, Comprehensive Organic Transformations, 2^{nd} edition; Wiley-VCH: New York, 1999; Comprehensive Organic Synthesis, B. Trost and I. Fleming (Eds.) vol. 1-9 Pergamon: Oxford, 1991; Comprehensive Heterocyclic Chemistry, A. R. Katrizky and C. W. Rees (Eds.) vol. 1-9 Pergamon: Oxford, 1984; Comprehensive Heterocyclic Chemistry II, A. R. Katrizky and C. W. Rees (Eds) vol. 1-11, Pergamon: Oxford, 1996; and Organic Reactions, Wiley & Sons: New York, 1991, Volume 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to filtration, distillation, crystallization, chromatography, and the like. Such materials can be characterized using conventional means, including physical constants and spectra data.

Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvents such as THF, DMF, Et₂O, DME and toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times are given for illustration only. The structure and purity of all final products were ascertained by TLC, mass spectrometry, ¹H NMR and/or high-pressure liquid chromatography (HPLC). Chemical symbols have their usual meanings. The following abbreviations have also been used v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variable mentioned below have the meanings as provided above.

Compounds of the present invention can be prepared according to the following general methods as exemplified in Schemes 1-4. For example, a Knoevenagel type condensation between cyanoacetate II and appropriately substituted aldehyde III can provide α,β-unsaturated ester IV. Concomitant reduction of the alkene and the cyano groups in IV can be accomplished with reducing agents such as CoCl₂—NaBH₄. The resulting saturated amine can be better isolated after protection with for example an N—BOC to give derivative V. Saponification of ester V and coupling of the resulting acid with amine VI will provide protected aminoamide VII. Finally removal of the protecting group can provide the desired aminoamide VIII (Scheme 1).

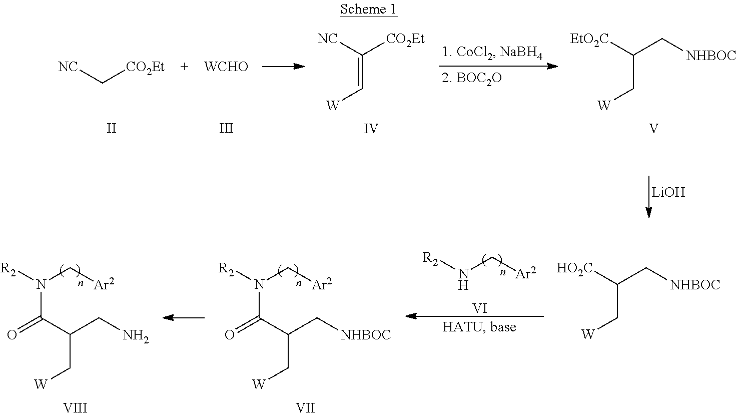

Alternatively the sequence can be modified with the initial coupling of amine VI with cyanoacetic acid IX to give amide precursor X (Scheme 2). Subsequent Knoevenagel condensation with substituted aldehyde III can deliver the α,β-unsaturated amide XI. Reduction of the double bond and the cyano group can again be accomplished with reducing agents such as CoCl₂—NaBH₄. The resulting saturated amine is most conveniently isolated as the N—BOC derivative VII. Finally, removal of the BOC protecting group under acidic conditions furnishes the desired aminoamide VIII.

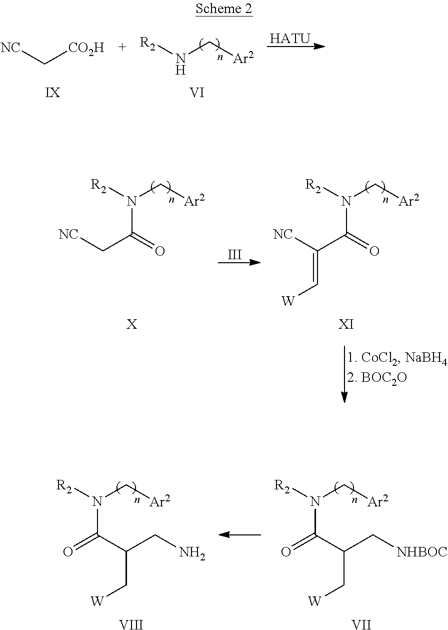

Aldehyde III and amine VI that are not commercially available can be readily accessed using the procedure described in patent application WO 2007/009250. In certain cases, it may be desirable to modify W in Scheme 1 and 2 prior to the final removal of the BOC protecting group. For example, the conversion of 2-chloropyridine XII into its corresponding pyridine XIII can be accomplished under typical hydrogenation conditions (Scheme 3).

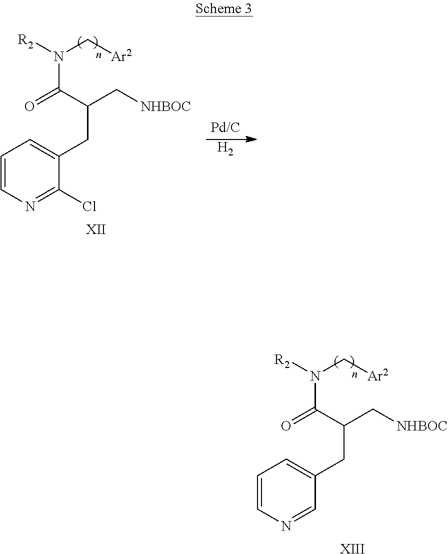

Also, the conversion of 2-bromopyridine XIV into 2-aminopyridine XV can be carried out with ammonium hydroxide in the presence of Cu₂O (Scheme 4).

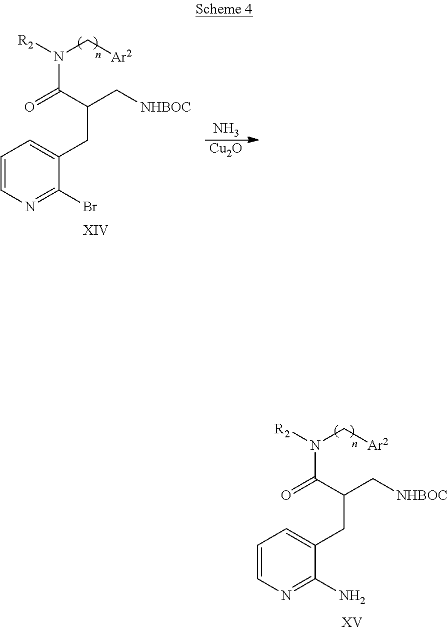

### EXAMPLE 1

### 3-amino-N-({2-chloro-5-[3-(methyloxy)propyl]phenyl}methyl)-N-cyclopropyl-2-(phenylmethyl)propanamide

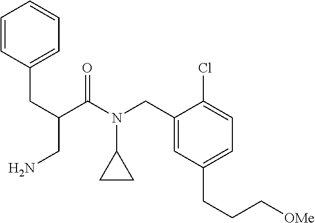

### Step 1: N-({2-chloro-5-[3-(methyloxy)propyl]phenyl}methyl)-2-cyano-N-cyclopropylacetamide

To a DMF solution (0.6 M) of cyanoacetic acid (1 eq.), Hunig's base (3 eq.) and N-({2-chloro-5-[3-(methyloxy)propyl]phenyl}methyl)cyclopropanamine (1 eq., WO 2007/009250) was added portionwise O-(7-azabenzotriazol-1-yl)-N,N,N,′N′-tetramethyluronium hexafluorophosphate (1.2 eq.). The resulting reaction solution was stirred at RT for 16 h. The now reddish solution was diluted with ether and washed with water and 10% aq. HCl. The aqueous washes were back-extracted with ether. The combined organic extracts were washed with brine, dried over Na₂SO₄, filtered and the filtrate concentrated in vacuo to afford a red semi-solid. Purification of the crude product thus obtained by way of column chromatography (SiO₂, 10:1 (v/v) Hex:EtOAc→1:4 (v/v) Hex:EtOAc) afforded the title compound as an off-white powder.

### Step 2: (2E)-N-({2-chloro-5-[3-(methyloxy)propyl]phenyl}methyl)-2-cyano-N-cyclopropyl-3-phenyl-2-propenamide

N-({2-Chloro-5-[3-(methyloxy)propyl]phenyl}methyl)-2-cyano-N-cyclopropylacetamide from the previous step (1 eq.) and benzaldehyde (1 eq.) were combined in benzene (0.05 M). To this solution was then added a few drops of piperidine and a Dean-Stark apparatus was attached to the reaction vessel. The resulting pale yellow solution was refluxed for 48 h. The volatiles were removed in vacuo and the crude product thus obtained was purified by way of column chromatography (SiO₂, 10:1 (v/v) Hex:EtOAc→1:4 (v/v) Hex:EtOAc). The title compound was isolated as a pale yellow solid.

### Step 3: 1,1-dimethylethyl [3-[({2-chloro-5-[3-(methyloxy)propyl]phenyl}methyl)(cyclopropyl)amino-3-oxo-2-(phenylmethyl)propyl]carbamate

(2E)-N-({2-Chloro-5-[3-(methyloxy)propyl]phenyl}methyl)-2-cyano-N-cyclopropyl-3-phenyl-2-propenamide from the previous step (1 eq.), cobalt(II) chloride hexahydrate (2 eq.) and di-tert-butyl dicarbonate (2 eq.) were combined in a 14:1 (v/v) EtOH:THF solution (0.07 M). To this mixture was then added sodium borohydride (10 eq.) slowly and portionwise at 0° C. The resulting black suspension was stirred at RT for 8 h. The volatiles were then removed in vacuo and the resulting residue was partitioned between EtOAc and 10% aq. HCl. The aqueous layer was separated and back-extracted with EtOAc. The combined organic extracts were then washed sequentially with 1 N aq. NaOH, water and brine, dried over MgSO₄, filtered and the filtrate concentrated in vacuo to afford a golden yellow oil. Purification of the crude product thus obtained by way of column chromatography (SiO₂, 10:1 (v/v) Hex:EtOAc→1:4 (v/v) Hex:EtOAc) afforded the title compound as a colorless oil.

### Step 4: 3-amino-N-({2-chloro-5-[3-(methyloxy)propyl]phenyl}methyl)-N-cyclopropyl-2-(phenylmethyl)propanamide

To a CH₂Cl₂ solution (0.5 M) of 1,1-dimethylethyl [3-[({2-chloro-5-[3-(methyloxy)propyl]phenyl}methyl)(cyclopropyl)amino-3-oxo-2-(phenylmethyl)propyl]carbamate from the previous step (1 eq.) was added HCl (4.0 M dioxane solution, 30 eq.). The resulting yellow solution was stirred at RT for 3 h. Following the removal of the volatiles in vacuo, the resulting residue was directly loaded onto a SiO₂ column packed with 97:3 (v/v) CH₂Cl₂:2.0 M NH₃ in MeOH. Elution with the same solvent system furnished the title compound as a colorless oil. MS (ESI+): 414.9.

### EXAMPLE 2

### 3-amino-N-({2-chloro-5-[3-(methyloxy)propyl]phenyl}methyl)-N-cyclopropyl-2-(1H-indol-4-ylmethyl)propanamide

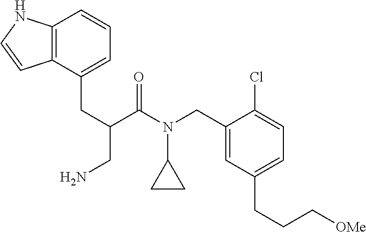

### Step 1: (2E)-N-({2-chloro-5-[3-(methyloxy)propyl]phenyl}methyl)-2-cyano-N-cyclopropyl-3-(1H-indol-4-yl) -2-propenamide

N-({2-Chloro-5-[3-(methyloxy)propyl]phenyl}methyl)-2-cyano-N-cyclopropylacetamide (1 eq., Example 1, Step 1) and 1H-indole-4-carbaldehyde (1.1 eq.) were combined in toluene (0.07 M). To this solution was then added a few drops of piperidine and a Dean-Stark apparatus was attached to the reaction vessel. The resulting pale yellow solution was refluxed for 18 h. The volatiles were removed in vacuo and the crude product thus obtained was purified by way of column chromatography (SiO₂, 7:3 (v/v) Hex:EtOAc→EtOAc). The title compound was isolated as a pale pink solid.

### Step 2: 1,1-dimethylethyl 4-{(1E)-3-[({2-chloro-5-[3-(methyloxy)propyl]phenyl}methyl)(cyclopropyl)amino]-2-cyano-3-oxo-1-propen-1-yl}-1H-indole-1-carboxylate

To a THF solution (0.06 M) of (2E)-N-({2-chloro-5-[3-(methyloxy)propyl]phenyl}methyl)-2-cyano-N-cyclopropyl-3-(1H-indol-4-yl)-2-propenamide (1 eq) from the previous step was added sodium hydride (3 eq.) at 0° C. The resulting red solution was stirred at 0° C. for 10 min and then at RT for 5 min. Finally, phenyl t-butyl carbonate (1.2 eq.) was added neat and the resulting solution was stirred at RT for 16 h. The reaction mixture thus obtained was diluted with ether and carefully quenched with water. The aqueous layer was separated and back-extracted with ether. The combined organic extracts were washed further with 1 N aq. NaOH, water and brine. The combined organic extracts were then dried over Na₂SO₄, filtered and the filtrate concentrated in vacuo. The crude product thus obtained was purified by way of column chromatography (SiO₂, 9:1 (v/v) Hex:EtOAc→EtOAc). The title compound was isolated as a bright yellow oil.

### Step 3: 1,1-dimethylethyl 4-{3-[({2-chloro-5-[3-(methyloxy)propyl]phenyl}methyl)(cyclopropyl)amino]-2-[({[(1,1-dimethylethyl)oxy]carbonyl}amino)methyl]-3-oxopropyl}-1H-indole-1-carboxylate

1,1-dimethylethyl 4-{(1E)-3-[({2-chloro-5-[3-(methyloxy)propyl]phenyl}methyl)(cyclopropyl)amino]-2-cyano-3-oxo-1-propen-1-yl}-1H-indole-1-carboxylate from the previous step (1 eq.), cobalt(II) chloride hexahydrate (2 eq.) and di-tert-butyl dicarbonate (2 eq.) were combined in EtOH (0.05 M). To this mixture was then added sodium borohydride (8 eq.) slowly and portionwise at 0° C. The resulting black suspension was stirred at RT for 8 h. The volatiles were then removed in vacuo and the resulting residue was partitioned between EtOAc and 10% aq. HCl. The aqueous layer was separated and back-extracted with EtOAc. The combined organic extracts were then washed sequentially with 1 N aq. NaOH, water and brine, dried over MgSO₄, filtered and the filtrate concentrated in vacuo to afford a pale green oil. Purification of the crude product thus obtained by way of column chromatography (SiO₂, 10:1 (v/v) Hex:EtOAc→EtOAc) afforded the title compound as a pale yellow oil.

### Step 4: 3-amino-N-({2-chloro-5-[3-(methyloxy)propyl]phenyl}methyl)-N-cyclopropyl-2-(1H-indol-4-ylmethyl)propanamide

To a CH₂Cl₂ solution (0.02 M) of 1,1-dimethylethyl 4-{3-[({2-chloro-5-[3-(methyloxy)propyl]phenyl}methyl)(cyclopropyl)amino]-2-[({[(1,1-dimethylethyl)oxy]carbonyl}amino)methyl]-3-oxopropyl}-1H-indole-1-carboxylate from the previous step (1 eq.) was added TFA (30 eq.×2). The resulting yellow solution was stirred at RT for 38 h. Following the removal of the volatiles in vacuo, the resulting residue was diluted with EtOAc and quenched with sat. aq. NaHCO₃. The aqueous wash was back-extracted with EtOAc and CH₂Cl₂. The combined organic extracts were washed further with brine, dried over Na₂SO₄, filtered and the filtrate concentrated in vacuo. Purification of the crude product thus obtained by way of column chromatography (SiO₂, 4:1 (v/v) CH₂Cl₂:2.0 M NH₃ in MeOH) afforded the title compound as a yellow oil. MS (ESI+): 454.2

### EXAMPLE 3

### 3-amino-N-({2-chloro-5-[3-(methyloxy)propyl]phenyl}methyl)-N-cyclopropyl-2-(1H-indol-6-ylmethyl)propanamide

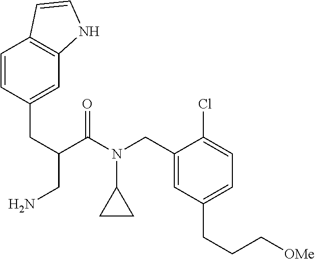

Prepared according to the procedure described in Example 2 but using instead 1H-indole-6-carbaldehyde as starting material. The title compound was obtained as a yellow oil. MS (ESI+): 454.4.

### EXAMPLE 4

### 3-amino-N-cyclopropyl-N-({3-{[2-(methyloxy)ethyl]oxy}-5-[3-(methyloxy)propyl]phenyl}methyl)-2-(3-pyridinylmethyl)propanamide

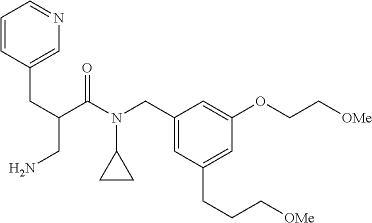

### Step 1: ethyl (2E)-3-({2-chloro-3-pyridinyl)-2-cyano-2-propenoate

Ethyl cyanoacetate (1 eq.) and 2-chloro-3-pyridinecarbaldehyde (1 eq.) were combined in toluene (0.2 M). To this solution was then added a few drops of piperidine and a Dean-Stark apparatus was attached to the reaction vessel. The resulting pale yellow solution was refluxed for 16 h. The volatiles were removed in vacuo and the crude product thus obtained was purified by way of column chromatography (SiO₂, 19:1 (v/v) Hex:EtOAc→3:7 (v/v) Hex:EtOAc). The title compound was isolated as a yellow solid.

### Step 2: ethyl 3-(2-chloro-3-pyridinyl)-2-[({[(1,1-dimethylethyl)oxy]carbonyl}amino)methyl]propanoate

Ethyl (2E)-3-({2-chloro-3-pyridinyl)-2-cyano-2-propenoate from the previous step (1 eq.), cobalt(II) chloride hexahydrate (2 eq.) and di-tert-butyl dicarbonate (2 eq.) were combined in EtOH (0.076 M). To this mixture was then added sodium borohydride (10 eq.) slowly and portionwise at 0° C. The resulting black suspension was stirred at RT for 14 h. The volatiles were then removed in vacuo and the resulting residue was partitioned between EtOAc and 10% aq. HCl. The aqueous layer was separated and back-extracted with EtOAc. The combined organic extracts were then washed sequentially with 1 N aq. NaOH, water and brine, dried over MgSO₄, filtered and the filtrate concentrated in vacuo to afford a golden yellow oil. Purification of the crude product thus obtained by way of column chromatography (SiO₂, 19:1 (v/v) Hex:EtOAc→3:7 (v/v) Hex:EtOAc) afforded the title compound as a colorless oil.

### Step 3: ethyl 3-({[(1,1-dimethylethyl)oxy]carbonyl}amino)-2-(3-pyridinylmethyl)propanoate

Ethyl 3-(2-chloro-3-pyridinyl)-2-[({[(1,1-dimethylethyl)oxy]carbonyl}amino)methyl]propanoate from the previous step (1 eq.) sodium acetate (1.3 eq.) and palladium (10% w/w on carbon, 0.1 eq.) were combined in MeOH (0.05 M). To this suspension was bubbled H₂ for 5 min and then allowed to stir under a static balloon atmosphere of H₂ at RT for 14 h. The insolubles were removed via filtration through a bed of celite and the filtrate concentrated in vacuo to afford a grey semi-solid. This residue was partitioned between EtOAc and water. The aqueous layer was separated and back-extracted with EtOAc. The combined organic extracts were then washed sequentially with brine, dried over MgSO₄, filtered and the filtrate concentrated in vacuo to afford a colorless oil. Further purification of the crude product thus obtained by way of column chromatography (SiO₂, 3:2 (v/v) Hex:EtOAc→EtOAc) afforded the title compound as a colorless oil.

### Step 4: 3-({[(1,1-dimethylethyl)oxy]carbonyl}amino)-2-(3-pyridinylmethyl)propanoic acid

To a 2:1 (v/v) THF:MeOH solution (0.63 M) of ethyl 3-({[(1,1-dimethylethyl)oxy]carbonyl}amino)-2-(3-pyridinylmethyl)propanoate from the previous step (1 eq.) was added LiOH (2.0 M aq. solution, 3 eq.). The resulting solution was stirred at RT for 10 h. Following the removal of the volatiles in vacuo, the residue was taken up in EtOAc and brought to a pH of 4 with 1 N aq. HCl. The aqueous layer was separated and back-extracted with EtOAc. The combined organic extracts were washed further with brine, dried over Na₂SO₄, filtered and the filtrate concentrated in vacuo to afford the title compound as a white solid.

### Step 5: 1,1-dimethylethyl[3-[cyclopropyl({3-{[2-(methyloxy)ethyl]oxy}-5-[3-(methyloxy)propyl]phenyl}methyl)amino]-3-oxo-2-(3-pyridinylmethyl)propyl]carbamate

To a DMF solution (0.1 M) of 3-({[(1,1-dimethylethyl)oxy]carbonyl}amino)-2-(3-pyridinylmethyl)propanoic acid from the previous step (1 eq.), Hunig's base (3 eq.) and N-({3-{[2-(methyloxy)ethyl]oxy}-5-[3-(methyloxy)propyl]phenyl}methyl)cyclopropanamine (1 eq., WO 2007/009250) was added portionwise O-(7-azabenzotriazol-1-yl)-N,N,N′,N′-tetramethyluronium hexafluorophosphate (1.2 eq.). The resulting reaction solution was stirred at RT for 16 h. The now reddish solution was diluted with EtOAc and washed with water and 1 N aq. NaOH. The aqueous washes were back-extracted with EtOAc. The combined organic extracts were washed with brine, dried over Na₂SO₄, filtered and the filtrate concentrated in vacuo to afford a red oil. Purification of the crude product thus obtained by way of column chromatography (SiO₂, 96:4 (v/v) CH₂Cl₂:2.0 M NH₃ in MeOH) afforded the title compound as a yellow oil.

### Step 6: 3-amino-N-cyclopropyl-N-({3-{[2-(methyloxy)ethyl]oxy}-5-[3-(methyloxy)propyl]phenyl}methyl)-2-(3-pyridinylmethyl)propanamide

To a CH₂Cl₂ solution (0.5 M) of 1,1-dimethylethyl[3-[cyclopropyl({3-{[2-(methyloxy)ethyl]oxy}-5-[3-(methyloxy)propyl]phenyl}methyl)amino]-3-oxo-2-(3-pyridinylmethyl)propyl]carbamate from the previous step (1 eq.) was added HCl (4.0 M dioxane solution, 30 eq.). The resulting yellow solution was stirred at RT for 3 h. Following the removal of the volatiles in vacuo, the resulting residue was directly loaded onto a SiO₂ column packed with 93:7 (v/v) CH₂Cl₂:2.0 M NH₃ in MeOH. Elution with the same solvent system furnished the title compound as a yellow oil. MS (ESI+): 456.2.

### EXAMPLE 5

### 3-amino-2-[(2-amino-3-pyridinyl)methyl]-N-({2-chloro-5-[3-(methyloxy)propyl]phenyl}methyl)-N-cyclopropylpropanamide

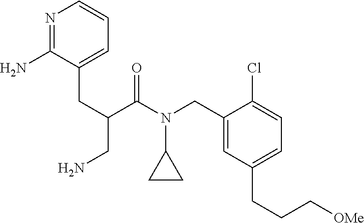

### Step 1: (2E)-3-(2-bromo-3-pyridinyl)-N-({2-chloro-5-[3-(methyloxy)propyl]phenyl}methyl)-2-cyano-N-cyclopropyl-2-propenamide

N-({2-Chloro-5-[3-(methyloxy)propyl]phenyl}methyl)-2-cyano-N-cyclopropylacetamide (1 eq., Example 1, Step 1) and 2-bromo-3-pyridonecarbaldehyde (1.1 eq.) were combined in toluene (0.05 M). To this solution was then added a few drops of piperidine and a Dean-Stark apparatus was attached to the reaction vessel. The resulting pale yellow solution was refluxed for 48 h. The volatiles were removed in vacuo and the crude product thus obtained was purified by way of column chromatography (SiO₂, 10:1 (v/v) Hex:EtOAc→EtOAc). The title compound was isolated as a yellow, viscous oil.

### Step 2: 1,1-dimethylethyl{2-[2-bromo-3-pyridinyl)methyl]-3-[({2-chloro-5-[3-(methyloxy)propyl]phenyl}methyl)(cyclopropyl)amino]-3-oxopropyl)carbamate

(2E)-3-(2-Bromo-3-pyridinyl)-N-({2-chloro-5-[3-(methyloxy)propyl]phenyl}methyl)-2-cyano-N-cyclopropyl-2-propenamide from the previous step (1 eq.), cobalt(II) chloride hexahydrate (2 eq.) and di-tert-butyl dicarbonate (2 eq.) were combined in 5:1 (v/v) EtOH:THF solution (0.055 M). To this mixture was then added sodium borohydride (10 eq.) slowly and portionwise at 0° C. The resulting black suspension was stirred at RT for 18 h. The volatiles were then removed in vacuo and the resulting residue was partitioned between EtOAc and 10% aq. HCl. The aqueous layer was separated and back-extracted with EtOAc. The combined organic extracts were then washed sequentially with 1 N aq. NaOH, water and brine, dried over MgSO₄, filtered and the filtrate concentrated in vacuo to afford a yellow oil. Purification of the crude product thus obtained by way of column chromatography (SiO₂, 10:1 (v/v) Hex:EtOAc→EtOAc) afforded the title compound as a colorless oil.

### Step 3: 1,1-dimethylethyl{2-[2-amino-3-pyridinyl)methyl]-3-[({2-chloro-5-[3-(methyloxy)propyl]phenyl}methyl)(cyclopropyl)amino]-3-oxopropyl)carbamate

In a sealed tube was combined 1,1-dimethylethyl{2-[(2-bromo-3-pyridinyl)methyl]-3-[({2-chloro-5-[3-(methyloxy)propyl]phenyl}methyl)(cyclopropyl)amino]-3-oxopropyl)carbamate from the previous step (1 eq.) and copper(I) oxide in ethylene glycol (0.06 M). To this was bubbled ammonia gas at 0° C. for 10 min before the vessel was sealed and heated at 80° C. for 18 h. The suspension was allowed to cool to RT, diluted with EtOAc and filtered through a bed of celite. The filtrate was then washed with 1 N aq. NaOH, water and brine, dried over Na₂SO₄, filtered and the filtrate concentrated in vacuo. Purification of the crude product thus obtained by way of column chromatography (SiO₂, 95:5 (v/v) CH₂Cl₂:2.0 M NH₃ in MeOH) afforded the title compound as a yellow oil.

### Step 4: 3-amino-2-[(2-amino-3-pyridinyl)methyl]-N-({2-chloro-5-[3-(methyloxy)propyl]phenyl}methyl)-N-cyclopropylpropanamide

To a CH₂Cl₂ solution (0.5 M) of 1,1-dimethylethyl{2-[(2-amino-3-pyridinyl)methyl]-3-[({2-chloro-5-[3-(methyloxy)propyl]phenyl}methyl)(cyclopropyl)amino]-3-oxopropyl)carbamate from the previous step (1 eq.) was added HCl (4.0 M dioxane solution, 30 eq.). The resulting yellow solution was stirred at RT for 4 h. Following the removal of the volatiles in vacuo, the resulting residue was directly loaded onto a SiO₂ column packed with 92:8 (v/v) CH₂Cl₂:2.0 M NH₃ in MeOH. Elution with the same solvent system furnished the title compound as a colorless oil. MS (ESI+): 431.1. Renin QFRET IC₅₀: 17 nM. Renin human plasma IC₅₀: 240 nM.

### Assays Demonstrating Biological Activity

### Inhibition of Human Recombinant Renin

The enzymatic in vitro assay was performed in 384-well polypropylene plates (Nunc). The assay buffer consisted of PBS (Gibco BRL) including 1 mM EDTA and 0.1% BSA. The reaction mixture were composed of 47.5 μl per well of an enzyme mix and 2.5 μl of renin inhibitors in DMSO. The enzyme mix was premixed at 4° C. and consists of the following components:
human recombinant renin (40 pM)
synthetic human angiotensin (1-14) (0.5 μM)
hydroxyquinoline sulfate (1 mM)
   The mixtures were then incubated at 37° C. for 3 h. The enzyme reaction was stopped by placing the reaction plate on wet ice.

To determine the enzymatic activity and its inhibition, the accumulated Ang I was detected by an enzyme immunoassay (EIA) in 384-well plates (Nunc). 5 μl of the reaction mixture or standards were transferred to immuno plates which were previously coated with a covalent complex of Ang I and bovine serum albumin (Ang I-BSA). 75 μL of Ang I-antibodies in assay buffer above including 0.01% Tween 20 were added and the plates were incubated at 4° C. overnight.

An alternative protocol could be used by stopping the enzymatic reaction with 0.02N final concentration of HCl. 5 μL of the reaction mixture or standards were transferred to immuno plates and 75 μL of Ang I-antibodies in assay buffer above including 0.01% Tween 20 were added and the plates were incubate at RT for 4 h.

The plates were washed 3 times with PBS including 0.01% Tween 20, and then incubated for 2 h at RT with an anti rabbit-peroxidase coupled antibody (WA 934, Amersham). After washing the plates 3 times, the peroxidase substrate ABTS ((2,2′-Azino-bis(3-ethylbenzthiazoline-6-sulfonic Acid)⁻ 2NH₃) was added and the plates incubated for 60 min at RT. The plate was evaluated in a microplate reader at 405 nm. The percentage of inhibition was calculated for each concentration point and the concentration of renin inhibition was determined that inhibited the enzyme activity by 50% (IC₅₀). The IC₅₀-values of all compounds tested were below 2 μM.

### Inhibition of Renin in Human Plasma

The enzymatic in vitro assay was performed in 384-well polypropylene plates (Nunc). The assay buffer consisted of PBS (Gibco BRL) including 1 mM EDTA and 0.1% BSA. The reaction mixture was composed of 80 μL per well of human plasma, enzyme, Ang I-antibodies mix and 5 μL of renin inhibitors in DMSO. The human plasma mix was premixed at 4° C. and consists of
human plasma from 10 normal donors
human recombinant renin (3 pM)
Ang I-antibodies.
   The mixtures were then incubated at 37° C. for 2 h.

To determine the enzymatic activity and its inhibition, the accumulated Ang I was detected by an enzyme immunoassay (EIA) in 384-well plates (Nunc). 10 μL of the reaction mixture or standards were transferred to immuno plates which were previously coated with a covalent complex of Ang I and bovine serum albumin (Ang I-BSA). 70 μL assay buffer were added and the plates were incubated at 4° C. overnight. The plates were washed 3 times with PBS including 0.01% Tween 20, and then incubated for 2 h at RT with an anti rabbit-peroxidase coupled antibody (WA 934, Amersham). After washing the plates 3 times, the peroxidase substrate ABTS ((2,2′-Azino-bis(3-ethylbenzthiazoline-6-sulfonic Acid)⁻ 2NH₃) was added and the plates incubated for 60 min at RT. The plate was evaluated in a microplate reader at 405 nm. The percentage of inhibition was calculated of each concentration point and the concentration of renin inhibition was determined that inhibited the enzyme activity by 50% (IC₅₀).

In vivo animal model—Female double transgenic rats were purchased from RCC Ltd, Füllingsdorf, Switzerland. All animals were maintained under identical conditions and had free access to normal pelleted rat chow and water. Rats were initially treated with enalapril (1 mg/kg/day) during 2 months. After approximately two weeks following cessation of enalapril treatment the double transgenic rats become hypertensive and reach mean arterial blood pressures in the range of 160-170 mmHg.

Transmitter implantation—The rats were anaesthetised with a mixture of 90 mg/kg Ketamin-HCl (Ketavet, Parke-Davis, Berlin FRG) and 10 mg/kg xylazin (Rompun, Bayer, Leverkusen, FRG) i.p. The pressure transmitter was implanted under aseptic conditions into the peritoneal cavity with the sensing catheter placed in the descending aorta below the renal arteries pointing upstream. The transmitter was sutured to the abdominal musculature and the skin closed.

Telemetry-System—Telemetry units were obtained from Data Sciences (St. Paul, Minn.). The implanted sensor consisted of a fluid-filled catheter (0.7 mm diameter, 8 cm long; model TA11PA-C40) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film to inhibit thrombus formation. The implants (length=2.5 cm, diameter=1.2 cm) weighted 9 g and have a typical battery life of 6 months. A receiver platform (RPC-1, Data Sciences) connected the radio signal to digitized input that was sent to a dedicated personal computer (Compaq, deskpro). Arterial pressures were calibrated by using an input from an ambient-pressure reference (APR-1, Data Sciences). Systolic, mean and diastolic blood pressure was expressed in millimeter of mercury (mmHg).

Hemodynamic measurements—Double transgenic rats with implanted pressure transmitters were dosed by oral gavage with vehicle or 10 mg/kg of the test substance (n=6 per group) and the mean arterial blood pressure was continuously monitored. The effect of the test substance is expressed as maximal decrease of mean arterial pressure (MAP) in the treated group versus the control group.

What is claimed is:

1. A compound of formula I, or a pharmaceutically acceptable salt thereof, or an optical isomer thereof, having the formula (I) 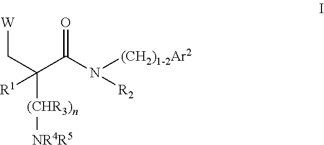
and pharmaceutically acceptable salts thereof, wherein
W is selected from the group consisting of
1) aryl, and
2) a heterocyclic ring system selected from the group consisting of:
a) a 5- or 6- membered saturated or unsaturated monocyclic ring with 1, 2, or 3 heteroatom ring atoms selected from the group consisting of N, O or S,
b) an 8-, 9- or 10-membered saturated or unsaturated bicyclic ring with 1, 2, or 3 heteroatom ring atoms selected from the group consisting of N, O or S, and
c) an 11- to 15-membered saturated or unsaturated triicyclic ring with 1, 2, 3, or 4 heteroatom ring atoms selected from the group consisting of N, O or S,
wherein said aryl or heterocyclic ring is unsubstituted, mono-substituted with R^{w}, disubstituted with groups independently selected from R^{w}, trisubstituted with groups independently selected from R^{w}, or tetrasubstituted with groups independently selected from R^{w}, and wherein any stable S or N heterocyclic ring atom is unsubstituted or substituted with oxo, said hetercyclic ring R^{w} substitutions being on one or more heterocyclic ring carbon or nitrogen atoms;
R^{w} is selected from the group consisting of
1) OH,
2) NH₂,
3) CN,
4) OCF₂H,
5) CF₃,
6) C₁-C₃alkyl,
7) C₁-C₃alkoxy,
8) —SO₂C₁-C₃alkyl, and
9) —SO₂H; and
n, in each instance in which it occurs, is independently 0, 1 or 2;
p, in each instance in which it occurs, is independently 0, 1 or 2;
R¹ and R³ are independently selected from the group consisting of H, C₁-C₆alkyl and C₂-C₆alkenyl, wherein the alkyl and alkenyl group is unsubstituted or substituted with one, two, three or four substituents independently selected from:
1) OH,
2) CN,
3) CF₃,
4) COOH,
5) C₁-C₆alkoxy,
6) C(O)R^{b},
7) C(O)N(R^{c})₂,
8) S(O)ₚC₁-C₆alkyl,
9) SO₂N(R^{c})₂,
10) N(R^{c})₂,
11) NHC(O)R^{b},
12) NHC(O)NHR^{d},
13) NHC(S)NHR^{d},
14) NH(NR^{c})NHR^{c},
15) tetrazolyl, and
16) —(CH₂)₁₋₂R^{e};
R⁴ is selected from the group consisting of H, C₁-C₆alkyl and C₂-C₆alkenyl, wherein the alkyl and alkenyl group is unsubstituted or substituted with one, two, three or four substituents independently selected from:
1) OH,
2) CN,
3) CF₃,
4) COOH,
5) C₁-C₆alkoxy,
6) C(O)R^{b},
7) C(O)N(R^{c})₂,
8) S(O)ₚC₁-C₆alkyl,
9) SO₂N(R^{c})₂,
10) N(R^{c})₂,
11) NHC(O)R^{b},
12) NHC(O)NHR^{d},
13) NHC(S)NHR^{d},
14) NH(NR^{c})NHR^{c}, and
15) tetrazolyl,
or R4, together with R5, forms a 5- or 6-membered heterocyclic ring which is unsubstituted or mono- or di-substituted with a substituent selected from the group consisting of ═O and C1-C6 alkyl;
R5 is selected from the group consisting of hydrogen and —C(NH(NH2), or R5, together with R4, forms a 5- or 6-membered heterocyclic ring which is unsubstituted or mono- or di-substituted with a substituent selected from the group consisting of ═O and C1-C6 alkyl;
R2 and Rb are independently selected from the group consisting of H, C1-C6alkyl, C3-C8cycloalkyl, C2-C6alkenyl, C1-C6alkoxy, CF3 and CH2CF3;
Rc is selected from the group consisting of H, C1-C6alkyl and CH2CF3;
Rd is selected from the group consisting of H and C1-C6alkyl, wherein the alkyl group is unsubstituted or substituted with one, two, three or four substituents selected from the group consisting of:
1) OH,
2) CN,
3) CF₃,
4) COOH, and
5) C(O)NHR^{c}, and
6) tetrazolyl;
R^{e} is a 5- or 6membered heteroaryl ring having 1 or 2 nitrogen atoms;
Ar² is independently selected from the group consisting of Ar¹ and a 9- or 10 membered fused bicyclic aryl or heteroaryl ring, wherein the fused bicyclic heteroaryl contains 1 to 4 heteroatoms selected from O, S and N, wherein the fused bicyclic aryl and heteroaryl are each unsubstituted or substituted with one, two, three or four substituents independently selected from the group consisting of:
1) OH,
2) CN,
3) halogen,
4) N₃,
5) NO₂,
6) COOH,
7) OCF₂H,
8) CF₃,
9) C₁-C₆alkyl, unsubstituted or substituted with Ar³,
10) C₁-C₆alkyl,
11) C₂-C₆alkenyl,
12) C₁-C₆alkoxy,
13) C(O)C₁ -C₆alkyl,
14) S(O)ₚC₁-C₆alkyl,
15) —O(CH₂)₁₋₂Ar³,
16) —O(CH₂)₁₋₂D,
17) —OC(O)D,
18) —OC(O)NH(C₁-C₆alkylene)C(O)NH₂, and
19) —OC(O)NH(C₁-C₆alkylene)(OH)R^{d};
wherein substituents (10)-(14) are unsubstituted or substituted with one, two three or four substituents independently selected from the group consisting of:
a) OH,
b) COOR^{d},
c) CN,
d) CF₃,
e) C₁-C₆alkoxy,
f) S(O)ₚC₁-C₆alkyl,
g) tetrazolyl
h) —C(O)NH₂,
i) —COONa,
j) —NR^{d}R^{d}, and
k) —NR^{d}C(O)R^{d};
heteroaryl ring containing 1 to 3 heteroatoms selected from O, S and N, wherein the substituted aryl ring and substituted heteroaryl ring are substituted with one, two three or four substituents independently selected from the group consisting of:
1) OH,
2) CN,
3) halogen,
4) N₃,
5) NO₂,
6) COOH,
7) OCF₂H,
8) CF₃,
9) C₁-C₆alkyl,
10) C₂-C₆alkenyl,
11) C₁-C₆alkoxy,
12) C(O)C₁-C₆alkyl, and
13) S(O)ₚC₁-C₆alkyl,
wherein substituents (9)-(13) are unsubstituted or substituted with one, two three or four substituents independently selected from the group consisting of:
a) OH,
b) COOH,
c) CN,
d) CF₃,
e) C₁-C₆alkoxy,
f) S(O)ₚC₁-C₆alkyl;
Ar¹ is an unsubstituted or substituted aryl ring or an unsubstituted or substituted 5- or 6-membered heteroaryl ring containing 1 to 3 heteroatoms selected from O, S and N, wherein the substituted aryl ring and substituted heteroaryl ring are substituted with one, two three or four substituents independently selected from the group consisting of:
1) OH,
2) CN,
3) halogen,
4) N₃,
5) NO₂,
6) COOH,
7) OCF₂H,
8) CF₃,
9) C₁-C₆alkyl,
10) C₂-C₆alkenyl,
11) C₁-C₆alkoxy,
12) C(O)C₁-C₆alkyl, and
13) S(O)ₚC₁-C₆alkyl,
wherein substituents (9)-(13) are unsubstituted or substituted with one, two three or four substituents independently selected from the group consisting of:
a) OH,
b) COOH,
c) CN,
d) CF₃,
e) C₁-C₆alkoxy,
f) S(O)ₚC₁-C₆alkyl;
Ar³ is an unsubstituted or substituted aryl ring or an unsubstituted or substituted 5- or 6-membered heteroaryl ring containing 1 to 3 heteroatoms selected from O, S and N, wherein the substituted aryl ring and substituted heteroaryl ring are substituted with one, two three or four substituents independently selected from the group consisting of:
1) OH,
2) CN,
3) OCF₂H,
4) CF₃,
5) C₁-C₃alkyl,
6) C₁-C₃alkoxy, and
7) —SO₂R^{d}; and
D is a 5- or 6-membered saturated heterocyclic ring having 1 or 2 nitrogen atoms and 0 or 1 oxygen atoms, wherein the ring may be unsubstituted or substituted with C₁-C₆alkyl.

2. A compound of claim 1, or a pharmaceutically acceptable salt thereof, wherein W is phenyl, pyridyl, indolyl, or pyridyl substituted with NH₂.

3. A compound of claim 1, or a pharmaceutically acceptable salt thereof, wherein R¹ is H.

4. A compound of claim 1, or a pharmaceutically acceptable salt thereof, wherein R³ is H.

5. A compound of claim 1, or a pharmaceutically acceptable salt thereof, wherein n is 1, R⁴ is H, and R⁵ is H.

6. A compound of claim 1, or a pharmaceutically acceptable salt thereof, wherein R² is cyclopropyl.

7. A compound of claim 1, or a pharmaceutically acceptable salt thereof, wherein Ar² is phenyl which is disubstituted with a group independently selected from Cl, —CH₂CH₂OCH₃, —OCH₂CH₂OCH₃, and —CH₂CH₂CH₂OCH₃.

8. A compound of claim 1, or a pharmaceutically acceptable salt thereof, selected from the group consisting of

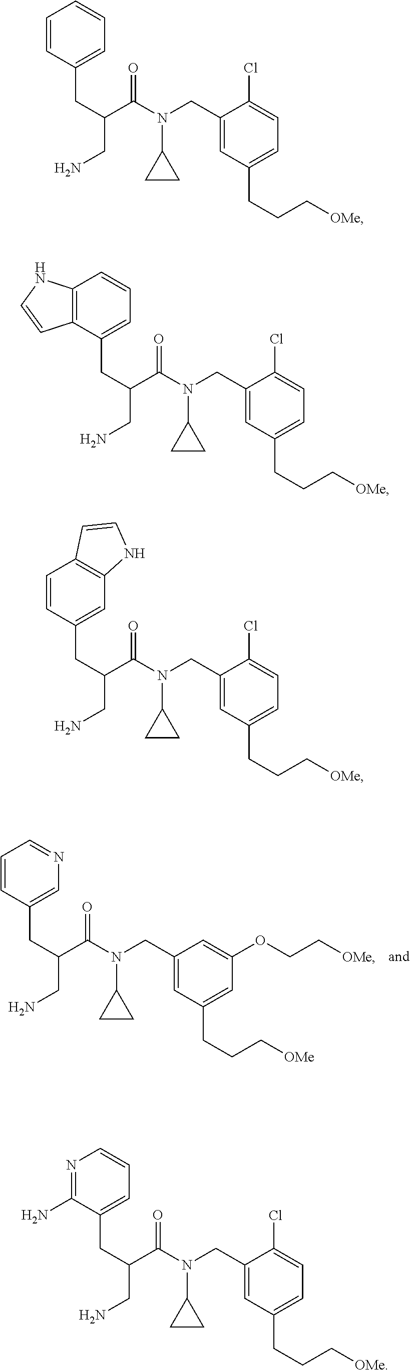

9. A pharmaceutical composition comprising an effective amount of a compound according to claim 1, or a pharmaceutically acceptable salt thereof, and a pharmaceutically acceptable carrier.

10. Use of a compound according to claim 1, or a composition according to claim 9, for the manufacture of a medicament for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

11. A method for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising the administration to a patient of a pharmaceutically active amount of a compound according to claim 1.
